(19) United States
(12) Patent Application Publication
(10) Pub. No.: US 2020/0276110 A1
(43) Pub. Date: Sep. 3, 2020

(54) A METHOD OF MANUFACTURING A VAGINAL RING

(71) Applicant: BAYER OY, Turku (FI)

(72) Inventor: Lasse KOIVULA, Turku (FI)

(21) Appl. No.: 16/063,773

(22) PCT Filed: Dec. 19, 2016
(86) PCT No.: PCT/EP2016/081701

§ 371 Date: Jun. 19, 2018

(30) Foreign Application Priority Data
Dec. 21, 2015 (EP) 15201507.9

(51) Int. Cl. A61K 9/00; A61K 45/06; B29D 99/00

(52) U.S. Cl. CPC A61K 9/0036; B29K 2083/00; B29D 99/0082; A61K 45/06

(57) ABSTRACT

The present invention relates to a method of manufacturing a vaginal ring, wherein the vaginal ring comprises at least one therapeutically active agent and a body comprising a crosslinked siloxane elastomer. The method comprises manufacturing the body in the form of a rod having a first end and a second end and forming the body into a ring by arranging an attachment part between the first end of the body and the second end of the body, wherein the attachment part comprises a non-crosslinked siloxane elastomer having a weight average molecular weight of 650-850 g/mol and a cross-linking catalyst, and curing the attachment part for a period of time of 1-30 second using a temperature of 125-220°.

### FIELD OF THE INVENTION

The present invention relates to a method of manufacturing a vaginal ring and to a vaginal ring manufactured by the present method.

### BACKGROUND OF THE INVENTION

Vaginal rings are typically used in contraception or for administering certain therapeutically active agents in a local manner. The vaginal rings are typically rings formed from a rod, i.e. a tubular form. The vaginal ring may be manufactured directly in the form of a ring, by moulding for example. A more convenient manner of manufacturing a vaginal ring is however to first manufacture the tube by for example extrusion and then form the ring by attaching ends of the tube to each other. A vaginal ring may also comprise several sections, for example each comprising a different therapeutically active agent. Furthermore, inactive segments can be present to give the ring a sufficient size to achieve a stabile fit in the uterine cavity. The sections are then attached to each other to form a ring. The attachment point is typically the weakest point in the ring, as the fact that the vaginal ring comprises a therapeutically active agent and all the materials used need to be biocompatible, limit the options for attaching the ends together.

Document U.S. Pat. No. 4,596,576 discloses a method for forming a release system in the form of a ring. In this document, the ends of the body parts are attached to each other by separate plugs made of an inert material. Such an attachment manner is however not very efficient in industrial production.

The ends of the body parts may also be attached to each other using an adhesive, such as silicon adhesive. There are however some technical problems when using adhesives. Indeed, the viscosity of the adhesives is typically too low, and the adhesive does not remain in place when the ends to be attached to each other are pressed together, leading to a too low strength of the attachment point. Moreover, two component adhesives typically dry very fast and hence their handling is difficult. On the other hand, the curing times of one component adhesives are too long for industrial use, as it would take too long a time for the attachment to become strong enough. Still further, the molar mass of the adhesives is low, making the adhesive sticky and difficult to handle.

There exists thus a need to provide a method for manufacturing a vaginal ring that overcomes the above problems and provides a fast and reliable way of forming the ring. The resulting attachment point should be strong enough for the use of the vaginal ring.

### OBJECTS AND SUMMARY OF THE INVENTION

An object of the invention is to provide a method of manufacturing a vaginal ring that is fast and reliable, suitable for industrial use. Another object is to provide a finished vaginal ring that is strong and does not break at the attachment point under a force it may be subject to when inserted, in use or removed. It is thus an object to at least partially overcome the problems encountered in prior art.

The present description relates to a method of manufacturing a vaginal ring, wherein the vaginal ring comprises
at least one therapeutically active agent and
a body comprising a crosslinked siloxane elastomer,
   the method comprising
manufacturing the body in the form of a rod having a first end and a second end,
forming the body into a ring by
   arranging an attachment part between the first end of the body and the second end of the body, wherein the attachment part comprises a non-crosslinked siloxane elastomer having a weight average molecular weight of 650-850 g/mol and a cross-linking catalyst, and
      curing the attachment part for a period of time of 1-30 second using a temperature of 125-2200.

The present description also relates to a vaginal ring obtainable by the present method.

### BRIEF DESCRIPTION OF THE DRAWING

FIG. 1 illustrates an embodiment of a machine usable in the present method.

FIG. 2 illustrates a vaginal ring according to an embodiment.

FIG. 3 illustrates the results of the tests of Examples 1-7.

### DETAILED DESCRIPTION OF THE INVENTION

The present description relates to a method of manufacturing a vaginal ring, wherein the vaginal ring comprises
at least one therapeutically active agent and
a body comprising a crosslinked siloxane elastomer,
   the method comprising
manufacturing the body in the form of a rod having a first end and a second end,
forming the body into a ring by
   arranging an attachment part between the first end of the body and the second end of the body, wherein the attachment part comprises a non-crosslinked siloxane elastomer having a weight average molecular weight of 650-850 g/mol and a cross-linking catalyst, and
curing the attachment part for a period of time of 1-30 second using a temperature of 125-2200.

The present method thus relates to a manufacturing method where the elastomer material used for attachment has a certain molecular weight. In addition, the method comprises manufacturing first the body in the form of a rod, i.e. a longitudinal piece. The cross-section of the rod is preferably either circular or elliptical, more preferably essentially circular. The body is thus not for example cast or injection moulded into a ring shape. The body may be manufactured for example by extrusion or injection moulding. In some embodiment, this step of the manufacturing method comprises forming a continuous rod of the body material and then cutting it to pieces having an appropriate length. Each piece is then formed to a ring form by attaching the ends together by the present method, using a defined curing temperature and a defined curing time. By curing in this description, it is meant polymerisation of the material, or hardening of the material, or cross-linking of the material, depending on the nature of the material. The period required for curing is also called curing time and the temperature required for curing is also called curing temperature. By the term body, it is meant the main part of the vaginal ring. It may be made of a single material and have a uniform structure, or it may comprise various parts having different structures. For example, it may comprise a core and another part surrounding the core, such as a membrane, which surrounding part may have any thickness as desired.

The curing temperature is 125-220° C. and may have a n influence on the strength of the finished ring. The curing temperature may be for example from 125, 130, 135, 140, 145, 150, 155, 160, 165, 170, 175, 180, 185, 190, 195, 200, 205, 210 or 215° C. up to 130, 135, 140, 145, 150, 155, 160, 165, 170, 175, 180, 185, 190, 195, 200, 205, 210, 215 or 220° C. The curing time is 1-30 seconds. It may thus be for example from 1, 2, 3, 4, 5, 7, 10, 12, 15, 18, 20, 22, 25 or 27 seconds up to 2, 3, 4, 5, 7, 10, 12, 15, 18, 20, 22, 25, 27 or 30 seconds. The choice of the curing temperature and curing time depends on the result that is desired to be achieved in the final product, as well as on the therapeutically active agent used and its ability to stand heat. One suitable curing temperature range is for example 150-200° C. One suitable curing time range is for example 5-20 seconds.

The present method, when combined with the specific molecular weight of the elastomer material of the attachment part, enables the manufacturing of vaginal rings in an efficient and speedy manner, suitable for industrial use.

The elastomer material in the body and the attachment part is a siloxane elastomer. For example it may be poly(dimethyl siloxane), which is a material known per se. Other suitable examples are modified polysiloxanes, substituted with functional groups, such as fluoropropyl or poly(ethyle oxide) groups or poly(disubstituted) siloxanes, where the substituents are lower alkyl, preferably alkyl groups of 1 to 6 carbon atoms or phenyl groups. The said alkyl or phenyl may be substituted or unsubstituted. According to one embodiment, the siloxane-based elastomer of the body is selected from the group comprising poly(dimethylsiloxane) (PDMS); siloxane-based elastomers comprising 3,3,3 trifluoropropyl groups attached to the silicon atoms of the siloxane units (fluoro-modified polysiloxanes); siloxane-based elastomers comprising poly(alkylene oxide) groups, where the poly(alkylene oxide) groups are present as alkoxy-terminated grafts or blocks linked to the polysiloxane units by silicon-carbon bonds. Suitable polysiloxanes and modified polysiloxane elastomers are described, for example, in EP 0652738 B1, WO 00/29464 and WO 00/00550. Also polyethylene oxide block-polydimethylsiloxane copolymer (PEO-b-PDMS) may be used, as well as combinations of any of the above-mentioned materials. The present inventor has however noticed that when the body has been made of a cured trifluoropropyl methyl siloxane (where all possible groups were substituted by fluoropropyl groups) and the attachment part has been made of the same material, cured during the attachment, the resulting attachment was weak and did not fulfil the requirements set internally for the strength of the attachment.

The elastomer of the body and of the attachment part may be the same or different, provided that the crosslinked elastomer of the body can react with the non-crosslinked elastomer of the attachment part, when the attachment part is cured. For example, if the body has been manufactured from a platinum-crosslinked poly(dimethyl siloxane), it can be formed into a ring with either a platinum-crosslinkable poly(dimethyl siloxane) or a peroxide-crosslinkable poly(dimethyl siloxane). Furthermore, it is also possible to use a blend of different siloxane elastomers. The same applies to a membrane if one is used before forming the body into a ring.

According to an embodiment, the siloxane elastomer of the body and of the attachment part is poly(dimethyl siloxane) (PDMS) and the catalyst is a platinum catalyst or a peroxide catalyst, provided that when the body is made of peroxide crosslinked poly(dimethyl siloxane), the attachment part is peroxide curable poly(dimethyl siloxane).

In addition to having the above-defined molecular weight, the elastomer should naturally also be biocompatible, as the product is to be inserted into the vagina of the user. The elastomer of the body should also enable the diffusion of the therapeutically active agent in order for the vaginal ring to fulfil its function, or otherwise be suitable for the structure used for releasing the therapeutically active agent. Such materials are known in the art and are thus not described in more detail in this description. In the following, when reference is made to the elastomeric material or elastomer material of the body and/or the attachment part, a siloxane elastomer is meant.

The weight average molecular weight of the elastomer of the attachment part is 650-850 g/mol. The weight average molecular weight may be for example from 650, 680, 700, 725, 750, 780, 800 or 820 g/mol up to 680, 700, 725, 750, 780, 800, 820 or 850 g/mol.

The vaginal ring comprises a therapeutically active agent. According to an embodiment, the therapeutically active agent is dispersed in the silicon elastomer of the body. The therapeutically active agent may also be contained in a separate reservoir forming a core of the body part. Furthermore, the vaginal ring may comprise more than one body part, such as two, three, four or five body parts. Each body part may contain a different therapeutically active agent or two or more of the body parts may contain the same therapeutically active agent. The release rate of the therapeutically active agent from each body part may be the same or different.

The therapeutically active agent may be any agent suitable as such, i.e. suitable for local administration. Some examples of suitable therapeutically active agents are progestins, estrogens, aromatase inhibitors and non-steroidal anti-inflammatory drugs (NSAID).

The therapeutically active agent(s) may be selected from group comprising progestins; chlormadinone acetate (CMA); norgestimate (NGM); norelgestromin (NGMN); norethisterone (NET)/norethisterone acetate (NETA); etonogestrel (3-keto-desogestrel); nomegestrol acetate (NOMAc); demegestone; promegestone; drospirenone (DRSP); medroxyprogesterone acetate (MPA); cyproterone acetate (CPA); trimegestone (TMG); levonorgestrel (LNG); norgestrel (NG); desogestrel (DSG); gestodene (GSD) and dienogest (DNG). Levonorgestrel (LNG); desogestrel (DSG); gestodene (GSD) and dienogest (DNG) are being preferred.

According to one embodiment natural and synthetic estrogens, especially estradiol or its esters, for example estradiol valerate or other conjugated estrogens (CEEs=conjugated equine estrogens) are preferred as estrogens. Particularly preferable are ethinylestradiol and estrogen or their esters such as estradiol valerate or benzoate.

According to one embodiment selective aromatase inhibitors such as anastrozole (Arimidex®); exemestane (Aromasin®); fadrozole (Afema®); formestane (Lentaron®); letrozole (Femara®); pentrozole; vorozole (Rivizor®); and pharmaceutical acceptable salts thereof are suitable for use as aromatase inhibitor. Anastrozole is being preferred.

According to one embodiment non-selective Cox inhibitors as well as selective Cox 2 inhibitors are equally suitable as non-steroidal anti-inflammatory drugs (NSAID). Meloxicam, piroxicam, naproxen, celecoxib, diclofenac, tenoxicam, nimesulide, lornoxicamand and indomethacin are being preferred, and indomethacin and diclofenac are particularly preferred.

The curing is carried out by using a catalyst that induces the curing. The catalyst must naturally also be such that it is itself biocompatible and the curing does not form any side products that are non-biocompatible or such that they cannot be removed by further treatment (such as post-curing). According to an embodiment, the curing catalyst is selected from a group consisting of platinum catalyst and peroxide catalyst. A platinum catalyst system is typically called an addition curing system and a peroxide catalyst system is typically called a free radical curing system. Both systems are known as such and are suitable for medical use.

One possible peroxide initiator, which may be incorporated into the attachment part is 2,4-dichlorobenzoyl peroxide. The 2,4-dichlorobenzoyl peroxide so decomposes by heat, whereby only minimal insignificant traces, if any at all, of initiator is present in the final intravaginal ring. Other examples of suitable organic peroxide initiators for cross-linking of the adhesive material are dicumyl peroxide, di-tert-butyl peroxide, dibenzoyl peroxide, tert-butyl benzoate, bis(4-methylbenzoyl) peroxide, bis(o-monochlorobenzoyl) peroxide, bis(p-monochlorobenzoyl) peroxide, 2,5-dimethyl-2,5-di(tertbutylperoxy) hexane, 1,1-bis(tert-butylperoxy)-3,3,5-trimethylcyclohexane, 1,6-bis(tert-butylperoxycarboxy) hexane and 1,4-bis-(tert-butylperoxyisopropoxy) benzene.

According to an embodiment, the amount of curing catalyst is 0.5-5 weight-% of the total weight of the attachment part.

According to an embodiment, the manufacturing of the at least body comprises arranging an inert portion at the first end and the second end of the body or between two separate body parts. By inert in this context it is meant a material that does not interfere with the diffusion of the therapeutically active agent, and this is typically an elastomer that does not comprise any therapeutically active agent. According to a preferred embodiment, the inert portion is made of the same siloxane elastomer as the rest of the body, but simply does not contain any therapeutically active agent.

This manufacturing of such body can be carried out for example by using an extruder with two different inlets for the material, one for the elastomer comprising the therapeutically active agent and one for the inert material and alternating the feed through the two inlets. Another alternative is to manufacture, for example by extrusion, a rod made of the elastomer comprising the therapeutically active agent and another rod made of the inert elastomer (i.e. not comprising a therapeutically active agent). Both rods are then cut into pieces of suitable length, thus forming non-inert portions (comprising the therapeutically active agent) and inert portions (without the therapeutically active agent). Typically the pieces of inert material are significantly shorter than the pieces comprising the therapeutically active agent. In a further step, the inert portions are attached to the non-inert portions according to the present method, i.e. by applying a curing heat for a curing time. According to so an embodiment, an inert portion is attached at both ends of the non-inert portion. Thereafter, a ring is formed by attaching the inert portions to each other. A further method of manufacturing such body is by coating extrusion or co-extrusion, where several layer can be formed on one core rod.

In a still further embodiment, the body of the vaginal ring may further comprise a membrane encasing a core. Such a vaginal ring may be manufactured for example by dipping the formed ring in a solution comprising a resin of the membrane material, followed by curing of the resin. In another alternative, the core part (or parts) are encased in the membrane before forming the ring structure. This may be done by extrusion or for example by inserting the rod inside a tubular film, followed by shrinking the film to fit snugly around the body part(s). Furthermore the membrane can be attached to the core by swelling the membrane material in a suitable solvent (such as cyclohexane), insertion of the core and subsequent removal of the solvent. The membrane can be also attached by expanding a membrane tube (either by applying vacuum or pressurized air), and subsequent insertion of the core. Such methods are known in the art.

In a preferred embodiment, the various parts of the core are first attached to each other, before adding the membrane. It is also possible that the parts of the core are not attached to each other, but only held together with the membrane. The end parts of the core may, in this embodiment, be inert parts, i.e. not comprising any therapeutically active agent. Thus in case the membrane is in the form of a tubular film arranged on the rod, the only attachment that is carried out when the membrane is in place, may be the formation of the ring structure.

According to an embodiment, the membrane has essentially the same length as the core or combination of core parts. According to another embodiment, the membrane may be slightly longer than the core or combination of core parts, in which case the attachment part is preferably arranged inside the membrane before curing. When a membrane is used, its thickness is for example below 1 mm, for example 0.05-1 mm. The materials used for the membrane are preferably selected from the same siloxane elastomers as the materials of the other parts of the vaginal ring. When a membrane is used and is arranged on the core before forming the ring itself, the membrane is preferably also curable during the attachment step.

According to an embodiment, the curing is effected by subjecting a portion of the body to the curing temperature. Indeed, it may be beneficial, depending on the therapeutically active agent and its ability to stand heat, to heat only a small part of the body instead of the whole body. According to another embodiment, said portion of the body extends from the first end towards the second end and from the second end towards the first end for a distance that is 2-7% of the length of body. Typically the connection point is arranged in essentially the middle of this portion that is cured. It is also possible to heat only the attachment part. The distance from the first end towards the second end may be for example 2, 3, 4, 5, 6 or 7% of the length of the body (or body part), such as 2-4%, 3-6% or 5-7% of the length of the body. Similar considerations apply for the second end of the body. When there are more than one body part, the same may apply mutatis mutandis to each body part or only to a part of them. Moreover, when the body comprises more than one body part, the length mentioned here typically refers to the length of each body part.

According to an embodiment, the attachment part further comprises a filler. The amount of filler may be 15-45 weight-% of the total weight of the attachment part. Some suitable ranges of amount of the filler are for example from 15, 18, 20, 22, 25, 30, 35 or 40 weight-% up to 18, 20, 22, 25, 30, 35, 40 or 45 weight-% of the total weight of the attachment part. The amount of filler as well as its nature may have an effect of the strength of the final ring. The body may also contain a filler, if desired.

For example, when silicon dioxide (also called silica) is used as the filler, a content of about 20 weight-% leads to a tensile strength of about 25-35 N (depending on the curing time and temperature). On the other hand, when the amount of about 35 weight-% of silica was used, the tensile strength was about 80-100 N (again, depending on the curing time and temperature).

According to a further embodiment, the filler is selected from a group consisting of silicon dioxide and diatomaceous earth. A mixture of both can of course also be used. The different parts of the vaginal ring may also comprise other components, such as colour pigments, for example titanium dioxide or zinc dioxide.

According to an embodiment, the body comprises a first and a second part each having a first end and a second end, and the method comprises attaching the first end of the first part to the first end of the second part and attaching the second end of the first part to the second end of the second part. The body may naturally also comprise a third, fourth, fifth part, and so on. The manufacturing process does not differ in essence from that described above.

According to a further embodiment, the body may comprise several parts that are not attached to each other, but are all together encased in a membrane. In this case, the ring is formed by attaching the ends of the body to each other, as described above. This embodiment can be used for example when the therapeutically active agent is such that it cannot be heated.

The present description also relates to a vaginal ring obtainable by a method described above. The manufacturing method, especially the attachment step has a significant impact on the strength of the finished ring. Some test results are given below in the experimental section. In some cases, the attachment point was even stronger than the remainder of the ring, i.e. the ring broke at a point different from the attachment point. According to the present applicant, a vaginal ring should be able to support a tensile strength of approximately 12 Nm. This strength requirement is the same as used for T-shaped intrauterine contraceptives comprising a copper wire.

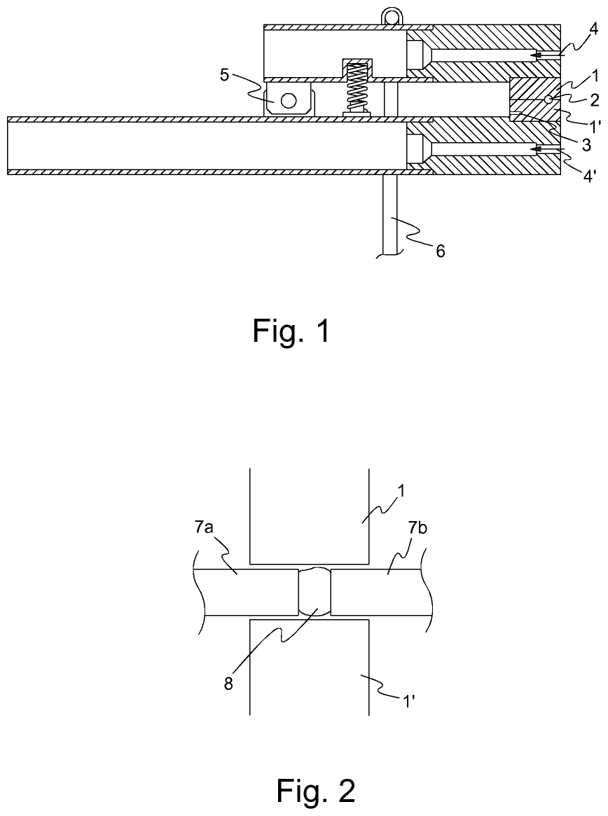

The method according to the present invention may be carried out by any suitable machine. One example of a suitable machine is illustrated in FIG. 1, explained in more detail below. When the method is carried out heating only a part of the material, the machine may comprise jaws or similar structure, than can be heated and which temperature can be controlled. The jaws may be manufactured in any suitable material, such as metal. One preferred material is steel, due to the facility of its sterilization. The width of the jaws may be for example about 10 mm, and the opening left for the material of the vaginal ring, when the jaws are in contact with each other, is selected such that it is suitable for the ring in question. According to one example, the diameter of the rod forming the ring is about 3-5 mm, for example 4.8 mm. In such case, the opening may have a diameter of 4.7-4.8 mm, for example. Indeed, the opening has a diameter that is essentially equal to the diameter of the rod to be formed into a ring, or slightly smaller.

### DETAILED DESCRIPTION OF THE DRAWING

FIG. 1 illustrates an embodiment of a machine usable in the present method. The machine comprises two jaw parts 1 and 1′, suitable to be in contact with each other and each comprising a groove, such that when the jaw parts are facing each other and in contact with each other, the grooves form a cylindrical opening 2 for the body of the vaginal ring. One of the jaw parts comprises a thermo element 3 and a heating element 4, 4′ is arranged on the outer side of each jaw part. The machine also comprises a hinge 5 allowing easy opening of the jaw and means for actioning 6 the opening of the jaws.

FIG. 2 illustrates a part of the machine of claim 1 in more detail, from another side. The Figure shows the two jaw parts 1 and 1′ as well as the first end portion 7a of the body part and a second end portion 7b of the body part. An attachment part 8 has been arranged between the two ends of the body part. For sake of clarity, some space has been left between the various parts.

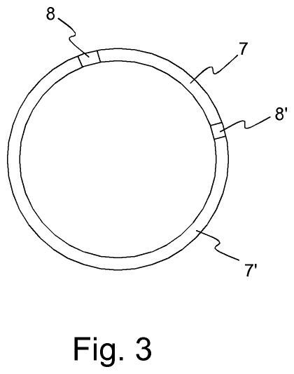

FIG. 3 illustrates a vaginal ring according to an embodiment. In this embodiment the ring comprises two body parts 7, 7′ and hence two attachment areas 8, 8′. The attachment areas are in reality not clearly demarked but the lines around the attachment areas 8, 8′ have been added for sake of clarity.

### EXPERIMENTAL PART

The following Examples were carried out in order to test vaginal rings manufactured according to the present method. When platinum curing was used, the elastomer was GEL1-9663-40 from Nusil and comprised 20 weight-% of silicon dioxide as well as the catalyst (amount as incorporated by the manufacturer). When peroxide was used as a catalyst, the elastomer was 70001 silicone elastomer SP70-011 from Dow Corning, comprising the catalyst (amount as incorporated by the manufacturer) and about 37 weight-% of silicon dioxide (as indicated by the manufacturer). In each example, the length of the body part was 160 mm. The diameter of the body part was 5 mm in all examples except for Example 8 (the ageing test). In Example 8, the rods comprised a membrane made of the peroxide-comprising elastomer (by Dow) having a thickness of 0.35 mm, while the diameter of the body part and the membrane together was 5 mm. In all examples, the length of the attachment part was 1 mm. Unless expressly specified, the body parts were made of the same material than the attachment parts.

The strength of the vaginal rings was tested by using the method of ASTM D1414 (1999), which is the standard test method for rubber O-rings. The tests were carried out by a Universal mechanical testing machine at 23±2° C. at 50±10% relative humidity. The test speed was 500 mm/min, load cell 500-1000 N. The test differed form that of the standard in that the diameter of the rods of the jig were 8 mm, not 9 mm or more as is mentioned in the standard. In all tests the vaginal ring was positioned in such a manner that the attachment part was on the side, in the middle. It was however noticed that the position of the attachment part in the test did not have any effect on the test results.

### Example 1

The vaginal ring was formed by attaching the two ends of the body part to each other, using a curing temperature of 175° C., a curing time of 15 seconds and a platinum catalyst. Five parallel samples were prepared and tested, and a mean value of the maximum load and extension at break calculated.

### Example 2

Example 1 was repeated with the exception that the curing time was 10 seconds.

### Example 3

Example 1 was repeated with the exception that the curing time was 5 seconds.

### Example 4

Example 1 was repeated with the exception that the curing temperature was 150° C. (curing time 15 s).

### Example 5

Example 4 was repeated with the exception that the curing time was 10 seconds.

### Example 6

Example 4 was repeated with the exception that the curing time was 5 seconds.

### Example 7

Example 2 was repeated (curing temperature 175° C., curing time 10 s) with the exception that the both platinum and peroxide catalysts were used, such that the body was platinum cured and the attachment part peroxide cured.

The results of Examples 1-7 are shown in Table 1 below, together with the curing times and temperatures.

TABLE 1

| Example | Curing time (s) | Curing temperature (° C.) | Max load (N) |
|---|---|---|---|
| 1 | 15 | 175 | 34.04 |
| 2 | 10 | 175 | 31.12 |
| 3 | 5 | 175 | 29.45 |
| 4 | 15 | 150 | 26.96 |
| 5 | 10 | 150 | 24.58 |
| 6 | 5 | 150 | 26.73 |
| 7 | 10 | 175 | 32.27 |

### Example 8

A vaginal ring according to an embodiment was also tested against aging, using the peroxide-cured elastomer. The test was carried out with six parallel samples. The attachment, i.e. formation of a ring, was carried out at a curing temperature of 150° C. and curing time 15 s, using a peroxide catalyst. The materials were the same as above and the rod comprised a membrane surrounding the body part as explained above.

The aging was carried out for a time period of six months. One set of samples was aged at 25° C. and 60% relative humidity (RH) (norm al conditions). Another set of samples was aged at 40° C. and a relative humidity of 75%. The latter conditions correspond to an accelerated aging test, and simulate an ageing for 24 months in normal conditions.

The results for Example 8 were as shown in Table 2 below.

TABLE 2

Max Load (N)

| | 25° C./60% RH | 40° C./75% RH |
|---|---|---|
| Example 8 | 90 | 78 |

As can be seen, the results after accelerated aging did not significantly differ from those of the normal aging and after both aging conditions, the vaginal ring had very satisfactory strength properties.

### Example 9

Some further tests were carried out. The materials used were the peroxide cured material mentioned above. The curing temperature was 140° C. and the curing time 20 seconds. Six parallel samples were prepared and tested, and a mean value of the results calculated.

### Example 11

Example 10 was repeated, except that the curing temperature was 150° C., the curing time 10 seconds and only two parallel samples were prepared.

### Example 12

Example 11 was repeated (curing temperature 150° C.), except that the curing time 15 seconds.

### Example 13

Example 12 was repeated (curing temperature 150° C., curing time 15 s), except that the samples contained titanium dioxide in an amount of 0.3 weight-% of the total weight.

### Example 14

Example 11 was repeated (curing temperature 150° C.), except that the curing time 20 seconds.

### Example 15

Example 10 was repeated, except that the curing temperature was 175° C., the curing time 5 seconds and only five parallel samples were prepared.

### Example 16

Example 15 was repeated (curing temperature 175° C.), except that the curing time 10 seconds.

### Example 17

Example 16 was repeated (curing temperature 175° C., curing time 10 s), except that the samples contained titanium dioxide in an amount of 0.3 weight-% of the total weight.

The results of Examples 10-17 are shown in Table 3 below, together with the curing times and temperatures.

TABLE 3

| Example | Curing time (s) | Curing temperature (° C.) | Max load (N) |
|---|---|---|---|
| 10 | 20 | 140 | 88.09 |
| 11 | 10 | 150 | 25.95 |
| 12 | 15 | 150 | 79.73 |
| 13 | 15 | 150 | 92.95 |
| 14 | 20 | 150 | 100.71 |
| 15 | 5 | 175 | 34.91 |
| 16 | 10 | 175 | 94.25 |
| 17 | 10 | 175 | 93.59 |

As can be seen, all the Examples give quite similar results for the breaking force test, although Examples 11 and 15 give significantly poorer results, probably meaning that the curing time for that particular curing temperature is not sufficient.

1. A method of manufacturing a vaginal ring, wherein the vaginal ring comprises
at least one therapeutically active agent and
a body comprising a crosslinked siloxane elastomer,
the method comprising
manufacturing the body in the form of a rod having a first end and a second end,
forming the body into a ring by
arranging an attachment part between the first end of the body and the second end of the body, wherein the attachment part comprises a non-crosslinked siloxane elastomer having a weight average molecular weight of 650-850 g/mol and a cross-linking catalyst, and
curing the attachment part for a period of time of 1-30 second using a temperature of 125-220° C.

2. A method according to claim 1, wherein the manufacturing of the body comprises attaching at least two body parts, in the form of a rod, together by arranging an attachment part between the body parts, wherein the attachment part comprises a non-crosslinked siloxane elastomer having a weight average molecular weight of 650-850 g/mol and a cross-linking catalyst and curing the attachment part for a period of time of 1-30 second using a temperature of 125-220° C.

3. A method according to claim 1, further comprising curing the attachment part for a period of time of 5-20 seconds.

4. A method according to claim 1, further comprising curing the attachment part using a temperature of 150-200° C.

5. A method according to claim 1, wherein the curing is effected by subjecting a portion of the body to the curing temperature.

6. A method according claim 1, wherein said portion of the body extends from the first end towards the second end and from the second end towards the first end for a distance that is 2-7% of the length of body.

7. A method according to claim 1, wherein the curing catalyst is selected from a group consisting of platinum catalyst and peroxide catalyst.

8. A method according to claim 1, wherein in the amount of curing catalyst is 0.5-5 weight-% of the total weight of the attachment part.

9. A method according to claim 1, wherein the siloxane elastomer of the body and of the attachment part is poly(dimethyl siloxane) and that the catalyst is a platinum catalyst or a peroxide catalyst, provided that when the body is made of peroxide crosslinked poly(dimethyl siloxane), the attachment part is peroxide curable poly(dimethyl siloxane).

10. A method according to claim 1, wherein the vaginal ring further comprises a membrane encasing the body.

11. A method according to claim 1, wherein the attachment part further comprises a filler.

12. A method according to claim 11, wherein the amount of filler is 15-45 weight-% of the total weight of the attachment part.

13. A method according to claim 11, wherein the filler is selected from a group consisting of silicon dioxide and diatomaceous earth.

14. A method according to claim 1, wherein the therapeutically active agent is selected from a group consisting of progestins, estrogens, aromatase inhibitors and non-steroidal anti-inflammatory drugs.

15. A vaginal ring obtainable by a method of claim 1.
